SEQUENCE LISTING (2 sequences)

SEQ ID NO: 1 - DNA, 2048 bp, Artificial Sequence
  Human Factor XII
ctattgatctggactcctggataggcagctggaccaacggacggacgccatgagggctctgctgctcctggggttcctgctggtgagcttggagtcaacactttcgattccaccttgggaagcccccaaggagcataagtacaaagctgaagagcacacagtcgttctcactgtcaccggggagccctgccacttccccttccagtaccaccggcagctgtaccacaaatgtacccacaagggccggccaggccctcagccctggtgtgctaccacccccaactttgatcaggaccagcgatggggatactgtttggagcccaagaaagtgaaagaccactgcagcaaacacagcccctgccagaaaggagggacctgtgtgaacatgccaagcggcccccactgtctctgtccacaacacctcactggaaaccactgccagaaagagaagtgctttgagcctcagcttctccggtttttccacaagaatgagatatggtatagaactgagcaagcagctgtggccagatgccagtgcaagggtcctgatgcccactgccagcggctggccagccaggcctgccgcaccaacccgtgcctccatgggggtcgctgcctagaggtggagggccaccgcctgtgccactgcccggtgggctacaccggacccttctgcgacgtggacaccaaggcaagctgctatgatggccgcgggctcagctaccgcggcctggccaggaccacgctctcgggtgcgccctgtcagccgtgggcctcggaggccacctaccggaacgtgactgccgagcaagcgcggaactggggactgggcggccacgccttctgccggaacccggacaacgacatccgcccgtggtgcttcgtgctgaaccgcgaccggctgagctgggagtactgcgacctggcacagtgccagaccccaacccaggcggcgcctccgaccccggtgtcccctaggcttcatgtcccactcatgcccgcgcagccggcaccgccgaagcctcagcccacgacccggaccccgcctcagtcccagaccccgggagccttgccggcgaagcgggagcagccgccttccctgaccaggaacggcccactgagctgcgggcagcggctccgcaagagtctgtcttcgatgacccgcgtcgttggcgggctggtggcgctacgcggggcgcacccctacatcgccgcgctgtactggggccacagtttctgcgccggcagcctcatcgccccctgctgggtgctgacggccgctcactgcctgcaggaccggcccgcacccgaggatctgacggtggtgctcggccaggaacgccgtaaccacagctgtgagccgtgccagacgttggccgtgcgctcctaccgcttgcacgaggccttctcgcccgtcagctaccagcacgacctggctctgttgcgccttcaggaggatgcggacggcagctgcgcgctcctgtcgccttacgttcagccggtgtgcctgccaagcggcgccgcgcgaccctccgagaccacgctctgccaggtggccggctggggccaccagttcgagggggcggaggaatatgccagcttcctgcaggaggcgcaggtaccgttcctctccctggagcgctgctcagccccggacgtgcacggatcctccatcctccccggcatgctctgcgcagggttcctcgagggcggcaccgatgcgtgccagggtgattccggaggcccgctggtgtgtgaggaccaagctgcagagcgccggctcaccctgcaaggcatcatcagctggggatcgggctgtggtgaccgcaacaagccaggcgtctacaccgatgtggcctactacctggcctggatccgggagcacaccgtttcctgattgctcagggactcatctttccctccttggtgattccgcagtgagagagtggctggggcatggaaggcaagattgtgtcccattcccccagtgcggccagctccgcgccaggatggcgaggaactcaataaagtgctttgaaaatgctgag

SEQ ID NO: 2 - protein, 615 aa, Artificial Sequence
  Human Factor XII
MRALLLLGFLLVSLESTLSIPPWEAPKEHKYKAEEHTVVLTVTGEPCHFPFQYHRQLYHKCTHKGRPGPQPWCATTPNFDQDQRWGYCLEPKKVKDHCSKHSPCQKGGTCVNMPSGPHCLCPQHLTGNHCQKEKCFEPQLLRFFHKNEIWYRTEQAAVARCQCKGPDAHCQRLASQACRTNPCLHGGRCLEVEGHRLCHCPVGYTGPFCDVDTKASCYDGRGLSYRGLARTTLSGAPCQPWASEATYRNVTAEQARNWGLGGHAFCRNPDNDIRPWCFVLNRDRLSWEYCDLAQCQTPTQAAPPTPVSPRLHVPLMPAQPAPPKPQPTTRTPPQSQTPGALPAKREQPPSLTRNGPLSCGQRLRKSLSSMTRVVGGLVALRGAHPYIAALYWGHSFCAGSLIAPCWVLTAAHCLQDRPAPEDLTVVLGQERRNHSCEPCQTLAVRSYRLHEAFSPVSYQHDLALLRLQEDADGSCALLSPYVQPVCLPSGAARPSETTLCQVAGWGHQFEGAEEYASFLQEAQVPFLSLERCSAPDVHGSSILPGMLCAGFLEGGTDACQGDSGGPLVCEDQAAERRLTLQGIISWGSGCGDRNKPGVYTDVAYYLAWIREHTVS

(19) United States
(12) Patent Application Publication
(10) Pub. No.: US 2003/0073652 A1
(43) Pub. Date: Apr. 17, 2003

(54) Ex-vivo and in vivo factor XII gene therapy for hemophilia A and B

(76) Inventors: Harvey Pollard, Potomac, MD (US); Bette Pollard, Potomac, MD (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 09/858,909

(22) Filed: May 17, 2001

Related U.S. Application Data
(60) Provisional application No. 60/205,014, filed on May 17, 2000.

(51) Int. Cl. A61K 48/00; C12N 5/08; C07K 14/745

(52) U.S. Cl. 514/44; 435/372; 530/384

(57) ABSTRACT

Bypass activity for hemophilia A and B can be generated by natural or recombinant Factor VIIa. Factor XIIa when implanted into a guinea pig or monkey also facilitates the conversion of endogenous factor VII to VIIa, thereby providing bypass activity. Additionally, certain modified versions of Factor XII are known to be intrinsically active, with properties like Factor XIIa. Administration of unencapsulated Factor XIIa to a guinea pig causes a transient increase in plasma bypass activity. A continuous source of Factor XIIa, as provided by a gene therapy, is therapeutic for both Hemophilia A and B. There are three ways to provide for gene therapy. In each case, the gene for Factor XII (or Factor XIIa) can be introduced into the cell by the usual means, including, but not limited to, as naked DNA, as a DNA/lipid mixture, or as part of a viral vector system. In one manifestation, cells can be transfected with full length or modified versions of Factor XII, ex-vivo, and allowed to continuously express versions of recombinant Factor XII from unencapsulated recombinant cells implanted in the body of the patient. A second mechanism would require encapsulating the cells within the body. As a third mechanism of introducing Factor XIIa into the patient, full length or modified versions of the gene for human Factor XII can be directly administered in vivo. The advantage is provision of a universal gene therapy for hemophilia A and B, rather than separate gene therapies involving either Factor VIII (Hemophilia A) or Factor IX (Hemophilia B).

This application claims priority under 35 U.S.C. §1.119(e) to provisional application serial No. 60/205,014, filed May 17, 2000.

### FIELD OF THE INVENTION

The invention relates to the use of recombinant Factor XII and truncated or mutated forms thereof, in gene therapy for conversion of inactive Factor VII to its active form in the treatment of Hemophelia A and B.

### BACKGROUND OF THE INVENTION

Hemophilia A is a coagulation disorder caused by a deficiency in Factor VIII:C (Factor 8) (Bloom, 1991; Rosendaal et al, 1991; Thompson, 1991; Handin et al, 1994). Hemophilia B is a coagulation disorder caused by a deficiency of Factor IX. Most U.S. and European hemophiliacs are undertreated due to the extraordinary cost of recombinant Factor VIII:C, and the high incidence of “inhibitors” or auto-antibodies to Factor VIII:C in patients (Aledort, 1998). Not only do these antibodies inhibit Factor VIII activity, but they also have a catalytic destructive effect (Lacroix-Desmazes, et al, 1999). Between 10% (Yee, et al, 1999) and 25% (Prescott et al, 1997) of hemophiliacs have such inhibitors, and they cost 10-fold more than the average to treat due to more frequent hospitalizations (Goudemand, 1998). Attempts to induce immune tolerance to Factor VIII:C are not often successful. Initially, such antibody development was not as frequently reported for recombinant Factor VIII:C as for plasma-derived Factor VIII:C (see Seremetis et al, 1999). However, Prescott et al (1997) from the American Red Cross report that there is no significant difference between the two classes of patients. Given financial limitations, priorities have been established for access to therapy, in which HIV-positive and/or hepatitis C-positive hemophiliacs are less likely to be treated optimally (Giangrande, 1997).

Hemophilia A is conventionally treated by replacement with either donor plasma-derived or recombinant Factor VIII:C (Bloom, 1992; Sultan and Algiman, 1992; Nilsson, 1992; Hedner and Glazer, 1992; Kasper, 1991). Similar approaches are used for the less common hemophilia B. The most common and debilitating consequences of hemophilia A are joint destruction and intracranial bleeds. The frequency of these catastrophic events can be reduced by prophyactic administration of Factor VIII:C. However, recombinant Factor VIII:C is extraordinarily expensive, and is usually given whenever a bleeding episode occurs, rather than continuously on a true prophyactic basis. Indeed, a therapeutic level of recombinant Factor VIII:C is said to have been achieved when coagulation has returned to 5% of “normal”. A 1% level is also said by some to be therapeutic. Aledort (1996) has concluded that “financial underwriting of care for this chronic disease remains the major uncertainty.” Two years later, Aledort (1998) further concluded “programs for prophylaxis or immune tolerance induction are impossible for most patients”.

The Hemophilia Foundation supports the concept that continuous prophylactic use is best, but agrees that the current economics makes this difficult for all but the most advantaged (Skolnick, 1994). Yet, even the data on true continuous prophylaxis with the best reagents available for the so-called “most advantaged” are far from encouraging. For example, a case has been made that continuous prophylaxis with recombinant Factor VIII:C yields a more favorable outcome than treatment rendered whenever a bleeding episode occurs (Miners et al, 1998). However, the term “favorable” in the latter case means that of 179 patients studied in London, the number of bleeds per year only decreased from a mean of 23.5 (range=1-107) to a mean of 14 (range=0-45). As a consequence of the expense of recombinant Factor VIII:C governments and other health-related organizations have therefore systematically argued against its prophylactic use on the basis of cost vs. benefit analysis (Smith et al, 1996; Miners et al, 1997; Green and Akehurst, 1998; Szucs et al, 1998).

The most commonly used source of Factor VII:C is from donor plasma. The plasma is collected and the Factor VIII:C purified by affinity chromatography on an anti-Factor VIII:C column. The product is also tested for viruses such as HIV, hepatitis, parvovirus B19 and others, and the viruses, real or potential, then inactivated by a combination of heat, detergent and solvent treatment. As a byproduct of these treatments the Factor VIII:C activity is substantially decreased. Nonetheless, the probability of infection due to transfusion with the final product still can not be reduced to zero (Altman, 1996).

There is a large amount of Factor VII present in human plasma, which can be used as a continuous source of activated Factor VII, called Factor VIIa, to treat Hemophilia A (and B as well). However, until recently no method was available to catalyze the conversion in vivo. It was known that Factor VII could be converted in vitro into Factor VIIa by either activated Factor X, called Factor Xa (Giles et al, 19880; Osterud, 1990) or activated Factor XII, called Factor XIIa (Hageman Factor, Factor XIIa; Kisiel et al, 1977; Brose and Majerus, 1980). The disadvantage of Factor Xa is that phospholipids such as phosphatidylserine are also needed as cofactors for the reaction. However, Factor XIIa is able to produce Factor VIIa from Factor VII without phospholipids.

Studies from the inventor's laboratory have validated these findings in vitro. The inventors have also extended the study with Factor XIIa into in vivo conditions in guinea pigs and monkeys (Ton-That et al, 2000). In the latter studies, bypass activity around a human anti-Factor VIII:C antibody could be achieved for up to one month in the Rhesus monkey system.

### SUMMARY OF THE INVENTION

An object of the invention is a recombinant FXIIa-coated bead.

An object of the invention is a Factor XII-expressing cell generated by transfection with an expression vector construct containing a gene encoding a Factor XII protein, or a truncated or mutant form thereof, with Factor VII activating activity.

Another object of the invention is an implant containing a Factor XII-expressing cell capable of producing Factor VIIa from endogenous Factor VII, and thereby generating bypass activity around Hemophilia Associated Factor VIII deficiency.

Another object of the invention is a method of treatment comprising administering a recombinant Factor XII polypeptide to a recipient in need of such treatment comprising administering a recombinant FXIIa-coated bead, an implant containing a recombinant FXIIa-coated bead, a cell expressing recombinant FXII, an implant containing a cell expressing recombinant FXII, a naked DNA vector containing a gene encoding a FXII polypeptide or a truncated or mutated form thereof, or a liposome encapsulated DNA vector containing a gene encoding a FXII polypeptide or a truncated or mutated form thereof.

Another object of the invention is a method for treating a subject with a congenital deficiency for Factor XII comprising administering an expression vector containing a gene encoding a Factor XII polypeptide, or a truncated or mutated form thereof.

Further objects and advantages of the present invention will be clear from the description as follows.

### BRIEF DESCRIPTION OF THE FIGURES

FIG. 1. Ex-Vivo Gene Therapy. The Factor XII gene, or a modified form of this gene (1) is prepared either as the naked DNA, the recombinant viral form or as a lipid adduct (2). One of these forms is transferred or transfected into a recipient cell, and the cell isolated in a form expressing recombinant FXII (3). The cell is then injected into a recipient organism, where it expresses FXII in the circulation.

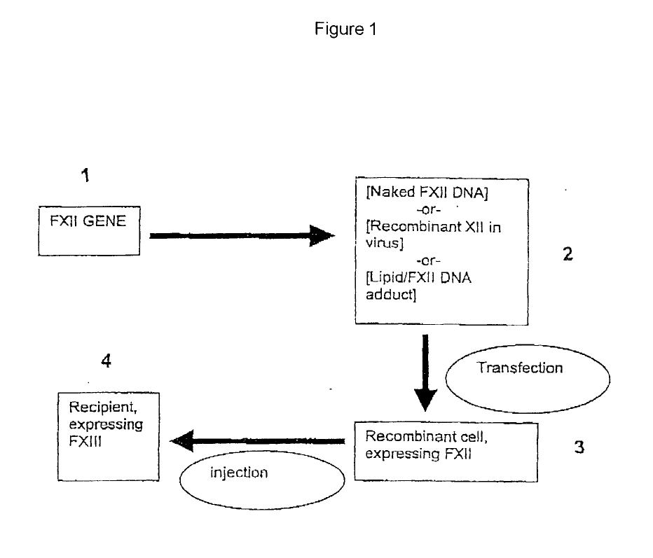

FIG. 2. Ex-Vivo Gene Therapy with Encapsulated Recombinant Cell. The Factor XII gene, or a modified form of this gene (1) is prepared either as the naked DNA, the recombinant viral form or as a lipid adduct (2). One of these forms is transferred or transfected into a cell, where it expresses Factor XII (3). The cell is placed in a porous chamber (4). The chamber is implanted in a recipient where Factor XII is expressed in the circulation (5).

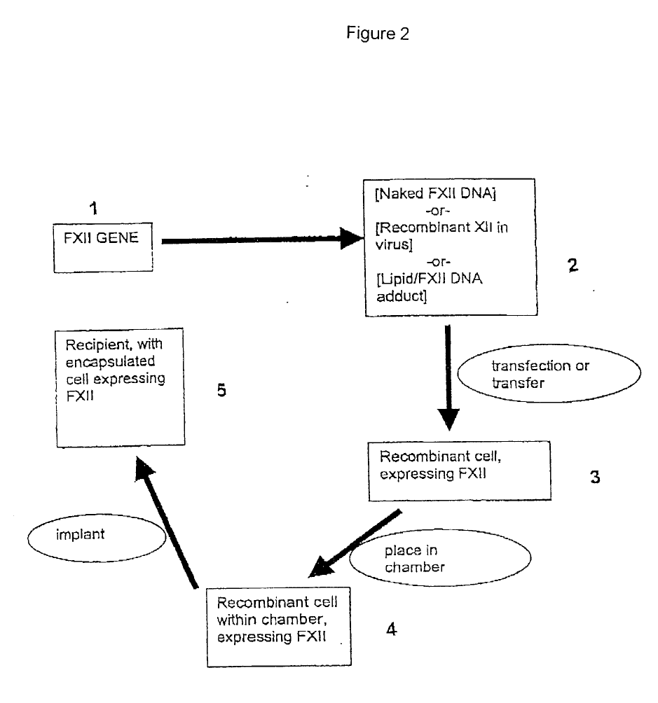

FIG. 3. In-Vivo Gene Therapy. The Factor XII gene, or a modified form of this gene (1) is prepared either as the naked DNA, the recombinant viral form or as a lipid adduct (2). One of these forms is transferred or transfected into a recipient organism, where it expresses FXII in the circulation.

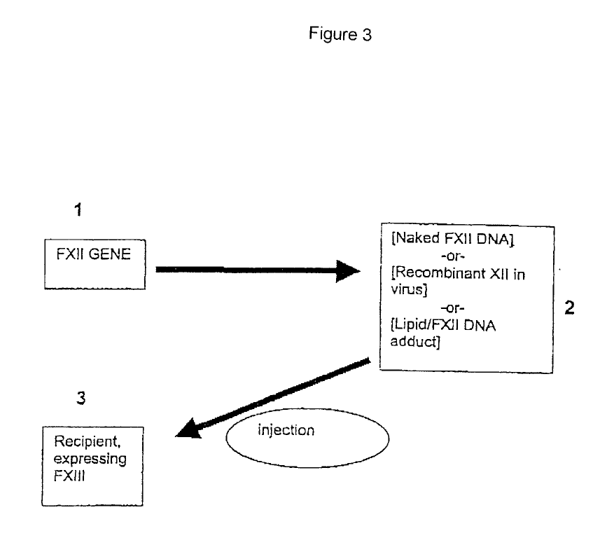

FIG. 4. Full length or modified recombinant Factor XII as a source of immobilized Factor XIIa inside an implant. Factor XII or modified Factor XII gene (1) is prepared for transfer to a cell for expression (2). The gene is transferred to an expression system (3), and the recombinant Factor XII species is assembled into a porous chamber (4). A porous chamber containing recombinant Factor XII is implanted into a recipient (5).

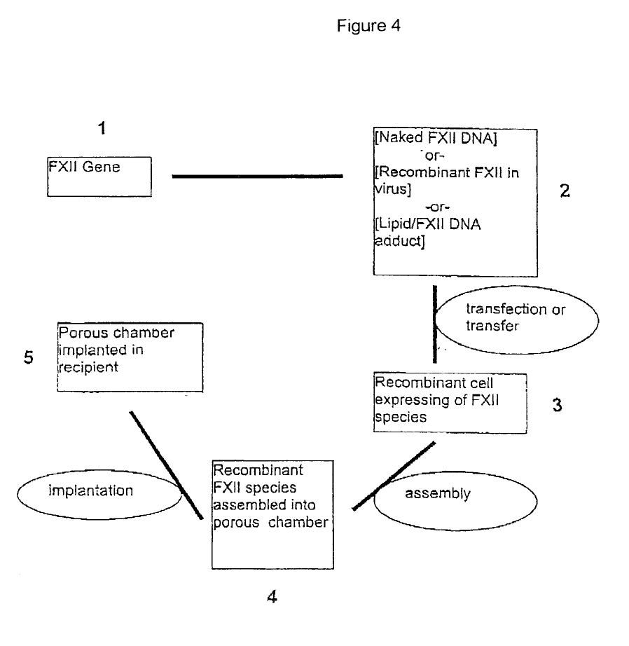

### DETAILED DESCRIPTION OF THE INVENTION

The administration of “activated prothrombin complex” (APC, or FEIBAd, ImmunoAG, Vienna, Austria) (Abildgaard et al, 1988; Kelly and Penner, 1976; Kurczynski and Penner, 1974; Lusher et al, 1980;) can be used to achieve coagulation in subjects with hemophelia. The improvement in coagulation state is believed to be due to high levels of Factor VIIa (Giles, 1987; Stewart et al, 1998). In 1983 purified Factor VIIa was used to treat two patients with Hemophilia A (Hedner and Kisiel, 1983; Hedner et al, 1989). Extensive clinical studies of recombinant Factor VIIa, manufactured by NOVO Nordisk (Bagsvaerd, Denmark), have verified the therapeutic value of this reagent for both Hemophilia A (Bell et al, 1993; Hedner, 1991; Schulman, 1992) and Hemophilia B (Schmidt et al, 1991; Brinkhous et al, 1989; Macik et al, 1993; Telgt et al, 1989). The mechanism by which Factor VIIa bypasses the Factor VIII:C and Factor IX steps in coagulation are not known. It was hypothesized that the Factor VIIa bypass activity occurred by Factor VIIa activation of the extrinsic pathway by complexing with Tissue Factor (Edgington et al, 1991). However, while this reaction certainly occurs, it turns out not to be the basis of the bypass reaction.

An additional advantage of Factor VIIa therapy is that many hemophiliacs have high levels of anti-Factor VIII:C antibodies (see above), which act to inhibit wild type or recombinant Factor VIII given therapeutically. The bypass activity provided by factor VIIa is completely unaffected by these anti-Factor VIII:C anti-antibodies. However, despite the attractiveness of the Factor VIIa approach to therapy of Hemophilia A, the use of recombinant Factor VIIa has been clinically compromised. First, cost considerations have made it available only to the most financially well off. Secondly, and perhaps more importantly, recombinant Factor VIIa has an extremely short half life in vivo (ca. 2 hours: Thomsen, et al, 1993; Hedner et al, 1993). The consequence is that in one of many reported recent examples of its use, 10 of 12 hemophilia A patients survived catastrophic intracranial hemorrhages by receiving an average of 97 injections each of recombinant Factor VIIa (“NovoSeven”) over a 15 day period (Arkin et al, 1998). Factor VIIa may offer advantages to the hemophiliac patient, however, the present outstanding need for a more efficacious and less costly form of recombinant factor VIIa mitigates against its practical use in the widespread treatment of hemophilia.

The present invention is based on the fact that hemophelia patients have normal levels of unactivated Factor VII, and that the patient's own blood contains a potential source of continuous and endogenous Factor VIIa. In order to access the patients own Factor VII and convert it to VIIa in an on-line manner, the present invention places an exogenous activator of Factor VII, in or near the blood stream, where it can activate Factor VII to VIIa, but in a manner which limits formation of thrombin.

In the present invention, the activator of Factor VII is a recombinant coagulation protein, preferably recombinant Factor XII. The ability of Factor XII to activate Factor VII to VIIa is well known and is described in a textbook of hematology (Hemostasis and Thrombosis, Robert W. Coleman, Jack Hirsh, Victor J. Marder, and Edwin W. Salzman, eds. J. B. Lippincott Co. Philadelphia and Toronto, page 9).

Human Factor XII was originally cloned and sequenced by Cool et al (1985) and by Que et al (1986) (SEQ ID No. 1). The protein comprised an N-terminal protease domain, a proline-rich hinge region, and five C-terminal regulatory domains (SEQ ID No. 2). Citarella et al (1992) reported that a full length recombinant Factor XII species of 80 kDa could be cloned into a vaccinia expression vector and expressed in HepG2 cells. This full-length species was similar to native Factor XII in all ways tested, and, upon activation, promoted coagulation with high efficiency.

Citarella et al (1992) also prepared a truncated Factor XII containing only the protease region and a portion of the proline-rich hinge domain (“FXII.Ipc”; amino acids residues 1-196 of SEQ ID No. 2). This 32 kDa species was described as unexpectedly displaying marked Factor XIIa-like protease activity, without need for preliminary activation.

The present invention concerns vector or plasmid constructs containing a gene encoding a full length polypeptide for FXII, or a truncated or a mutated form thereof. Most preferably, the protease activity is retained for a recombinant FXII polypeptide. A recombinant FXII polypeptide can range in size from the 80 kDa full length form of the protein (SEQ ID No. 2) to the 32 kDa form described by Citarella. Preferably, the FXII polypeptide comprises a sequence of amino acid residues 1 to 615, 1 to 515, 1 to 415, 1 to 315 or 1 to 215 of SEQ ID No. 2, and more preferably, a sequence of amino acid residues of about 1 to 196 of SEQ ID No. 2.

The term “gene” refers to a polynucleic acid or a nucleotide, which encodes a peptide, a prepeptide, a protein or a marker, or to a vector or plasmid containing such a polynucleic acid or nucleotide.

A “mutant” or “modified” gene or peptide refers to a gene having a sequence in that one or more bases or residues are deleted, substituted or added at any position therein, including either terminus.

An object of this invention is the delivery of these Factor XII-related sequences as in vivo or ex vivo gene therapies for treatment of hemophelia A and B.

The drawback of much gene therapy today is delivery of the therapeutic gene to a cell in vitro, more particularly to a cell in a living organism, and the level and duration of expression (Kaufman, 1999). Vectors that have been used in gene therapy applications related to hemophilias include adenovirus, retrovirus, adeno-associated virus (AAV), and naked DNA transfer (Pipe and Kaufman, 2000).

The adenoviral system with either full length Factor VII, or Factor VII from which the B-domain has been deleted, has been studied intensively. Expression levels using recombinant adenoviral vectors have been optimized in hepatocytes (Andrews et al, 1999), and studied when transfected into factor VII-deficient mice (Connelly et al, 1996; Connelly et al, 1998; Connelly et al, 1999). A minimal adenoviral vector, devoid of all viral genes, has been developed which theoretically avoids the intrinsic toxicity of the adenovirus (Balague et al, 2000). Such “mini-adenoviral” vectors have also been tested with Factor VIII in mice and dogs (Zhang et al, 1999). Ex-vivo gene therapy of primary fibroblasts with adenovirus mediated Factor VII has also been reported. The recombinant gene was placed into the virus in the test tube, and the gene-virus combination transfected into the cell. The cells were then implanted into the spleen of the recipient animal (Zatloukal et al, 1994). Adenovirus-mediated transfer of Factor IX has been associated with dose-limiting toxicity in monkeys (Lozier et al, 1999).

Adeno-associated virus (AAV) systems have been intensively developed because of the lack of intrinsic toxicity and long-term effects of their recombinant proteins. The problem with AAV systems, in addition to the difficulty in obtaining high titer preparations, has been their low capacity. To avoid this latter problem, one approach has been to transfect multiple vectors bearing the heavy and light chains of Factor VIII (Burton et al, 1999). Use of smaller promotors as well as truncated Factor VIII has been reported (Gnatenko et al, 1999). Tests with AAV in gene therapy with the smaller Factor IX have been increasingly successful. Long term expression of Factor IX in dogs has been achieved without detectable dissemination to the gonads (Monahan et al, 1998; Herzog et al, 1999). Kay et al (2000) have also tested AAV carrying the smaller factor IX in humans, with encouraging results.

Retroviruses have been used to transfer Factor VIII into bone marrow cells, ex vivo, in anticipation of using patient marrow as a target for gene therapy (Chiang et al, 1999). This approach has been used with Factor VIII deficient mice in hopes of generating both immune tolerance and therapeutic Factor VIII expression with transfected human Factor VIII (Evans and Morgan, 1998). A fraction of the animals responded positively. An increase in retrovirus mediated Factor VIII transfection efficiency has also been reported in rodents when regeneration of liver was initiated simultaneously with retroviral administration (Oh, et al, 1999). Approximately 50% success has been reported with IV injection of new born, Factor VIII-deficient mice with a human Factor VIII-retrovirus (VandenDreissche et al, 1999).

Novel therapeutic strategies include new recombinant products, engineered to have reduced immunogenicity (Voorberg et al, 1996; Pipe and Kaufman, 1997; and Barrow et al, 2000). Peptidomimetics have been considered, where protein subdomains of the clotting factors might have biological activity. Finally, specific gene repair strategies employing RNA/DNA oligonucleotides have been proposed for specifically targeted correction (Bandyyopadhyay et al, 1999).

In a preferred embodiment, a gene, a fragment thereof or a mutant thereof, encoding a proteolytically active polypeptide for FXII is subcloned into a viral or non-viral expression vector. The viral vector is selected from at least one vector obtained from an adenovirus, a retrovirus such as MMLV, a herpes simplex virus, a vaccinia virus or an adeno-associated virus (AAV), and more preferably an AAV vector. A non-viral vector is preferably an expression plasmid.

The gene that is to be introduced by the vector constructs can be in the form of a nucleic acid containing the FXII gene or a fragment thereof, or a mutant thereof which, if necessary, is provided with the appropriate regulatory regions such as promoters.

Preferably, the DNA construct contains a promoter to facilitate expression of the DNA of interest within a host cell. Preferably the promoter is a strong, eukaryotic promoter. Exemplary eukaryotic promoters for facilitating transcription in a eukaryotic cell include promoters from cytomegalovirus (CMV), mouse mammary tumor virus (MMTV), Rous sarcoma virus (RSV), and adenovirus. More specifically, exemplary promoters include the promoter from the immediate early gene of human CMV (Boshart et al., Cell 41:521-530, 1985) and the promoter from the long terminal repeat (LTR) of RSV (Gorman et al., Proc. Natl. Acad. Sci. USA 79:6777-6781, 1982). Of these two promoters, the CMV promoter is preferred as it provides for higher levels of expression than the RSV promoter.

For eukaryotic expression (e.g., in an endothelial cell), the construct preferably comprises at least a eukaryotic promoter operably linked to a DNA of interest, which is in turn operably linked to a polyadenylation sequence. The polyadenylation signal sequence may be selected from any of a variety of polyadenylation signal sequences known in the art. Preferably, the polyadenylation signal sequence is the SV40 late polyadenylation signal sequence. The construct may also include sequences in addition to promoters which enhance expression in host cells (e.g., enhancer sequences, introns). For example, the construct can include one or more introns, which can increase levels of expression of the DNA of interest, particularly where the DNA of interest is a cDNA (e.g., contains no introns of the naturally-occurring sequence). Any of a variety of introns known in the art may be used. Preferably, the intron is the human beta-globin intron and inserted in the construct at a position 5′ to the DNA of interest.

Other components such as a marker (e.g., an antibiotic resistance gene (such as an ampicillin resistance gene) or beta-galactosidase) to aid in selection of cells containing and/or expressing the construct (e.g., during the process of vector construction), an origin of replication for stable replication of the construct in a bacterial cell (preferably, a high copy number origin of replication), a nuclear localization signal, or other elements which facilitate production of the DNA construct, the protein encoded thereby, or both.

Techniques for production of nucleic acid constructs for expression of exogenous DNA or RNA sequences in a host are known in the art (see, for example, Kormal et al., Proc. Natl. Acad. Sci. USA, 84:2150-2154, 1987; Sambrook et al. Molecular Cloning: A Laboratory Manual, 2nd Ed., 1989, Cold Spring Harbor Laboratory Press, Cold Spring Harbor, N.Y.; each of which are hereby incorporated by reference with respect to methods and compositions for eukaryotic expression of a DNA of interest).

Another object of the invention is a transfected host cell containing a stably integrated vector construct containing a gene encoding a full length polypeptide for FXII, or a truncated or a mutated form thereof. In this case, the source of FXII is a host cell genetically engineered to express a proteolytically active form of FXII.

Examples of suitable host cells include eukaryotic cells, preferably mammalian cells, more preferably human cells, and most preferably cells obtained from the same subject or individual. Specific kinds of eukaryotic cells include but are not limited to blood cells, hepatocytes, fibroblasts, and endothelial cells.

The term “mammal” refers to a human being or an animal.

A non-viral vector construct can be transfected under in vitro conditions into a host cell by any one of the following methods including but not limited to calcium phosphate or DEAE-dextran precipitation, microinjection, electroporation, cationic liposomes, pH sensitive or negatively-charged liposomes, or ballistic DNA injection.

The activity for an FXII expressing cell can be measured by any clinically recognized assay, more preferably a COATEST assay.

An ex-vivo transfected host cell expressing a proteolytically active form of FXII can be administered to a recipient by intravenous, intraarterial, intraportal, intracranial, intrapleural, intraperitoneal or local introduction. The concentration of the recombinant protein expressed by the transfected cells is an effective amount whereby any inactive FVII contacting FXII is protealyzed into active FVIIa.

Cells expressing a recombinant protein, preferably FXII, which have been attached to a solid support, may also be placed in porous chambers as described in U.S. Pat. No. 5,908,399 and U.S. Pat. No. 6,174,299, and implanted in hemopheliac patients. The porous chamber comprises a permeable membrane having one or more walls defining a hollow chamber and a plurality of holes extending through the walls of the membrane and permitting fluid to enter and exit the chamber of the membrane. Each of the holes is sized so that it is large enough to permit inactive FVII to enter the chamber of the membrane and activated FVIIa to exit the chamber of the membrane, but small enough to prevent fibrinogen from entering the chamber of the membrane.

The porous chamber should possess a region that allows the chamber to be affixed to the abdominal wall, also having a design such as a diaphragm that would allow penetration of a syringe for removal or addition of materials therefrom, including but not limited to replacement of a solid support, a cell-coated support or a coagulation protein-coated support.

The solid support may comprise any solid matrix or particle such as a microcarrier bead, more preferably, a cytodex™ bead.

The permeable membrane may comprise any container having the property of being biologically compatible, and having holes being sized to permit inactive FVII to enter the chamber of the membrane and activated FVIIa to exit the chamber. Examples of membranes include but are not limited to a dialysis bag or a dialysis membrane, a geometric filter comprising polymethacrylate or polycarbonate, or a statistical hole filter comprising cellulose acetate. Preferably, the size of each of the holes in the membrane is less than about 100 angstroms, and most preferably 40-50 angstroms, which is suitable for a protein of about 50,000 daltons.

In the methods of the present invention, the membrane is implanted so that it is in contact with the subject's blood stream. In this regard, the membrane is filled with a solid support being bound to a recombinant coagulation protein, more preferably an FVII activator, or to a cell expressing an FVII activator, more preferably, FXIIa, sterilized and implanted using conventional medical equipment and procedures into the femoral vein, or intraperitoneally. The membrane may be removed by similar surgical procedures. Alternatively, a bypass device may be installed into the subject, and circulation allowed through an arteriovenous bypass (extracorporeal therapy).

The concentration of the recombinant protein expressed by the attached cells is an effective amount whereby any inactive FVII passing through the chamber is protealyzed into active FVIIa.

Purified, recombinant Factor XII-related sequences including the full-length protein, or a truncated or mutated form thereof, can be coupled to a solid support, which is then placed in the porous chamber or administered directly to a recipient.

The activity for an FXII coated-bead can be measured by any clinically recognized assay, more preferably a COATEST assay.

A solid support may comprise any material widely used for similar purposes in protein chemistry including but not limited to a 4% cross-linked beaded agarose, CNBr-activated Sepharose 4B, beaded acrylamide and porous glass beads.

A recombinant Factor XII protein can be purified by chemistries involving protein separation including fraction or immunoadsorption.

In a preferred embodiment, a purified coagulation protein, preferably a purified recombinant FXIIa is coupled to beads by mixing the protein with 750 μl of Affigel-10 beads (Pierce) in desalting buffer (0.1 M HEPES/NaOH buffer, pH7.4 and 0.3 M NaCl) adjusted to 80 mM CaCl₂ for 12 hr at 4° C. The beads are centrifuged at 12,000 g for 10 sec and washed three times in protein-free, calcium-free desalting medium. The unreacted sites are blocked by resuspending the beads for 24 hr at 4° C. in1M glycine in desalting buffer followed by washing in phosphate buffered saline.

Non-viral vector constructs can be employed as a pharmaceutical or a constituent of a pharmaceutical, with the vectors preferably being used for preparing a pharmaceutical for the intravenous, intraarterial, intraportal, intracranial, intrapleural, intraperitoneal or local introduction of a desired gene into specific target cells. Pharmaceutically acceptable carriers for the novel vectors are those listed in Remington's Pharmaceutical Sciences, Mack Publishing Co., Easton, Pa. 1988, hereby incorporated by reference.

The simplicity of injecting a naked plasmid DNA ranging in size from 2-20 kilobases into a target organ, tissue or cell with a syringe has greatly influenced many aspects of gene therapy research. Tissues which exhibit transgene expression following naked plasmid DNA injection include but are not limited to thymus, skin, cardiac muscle and skeletal muscle. Of these tissues, long-term transgene expression has been observed in striated muscle.

The number of injections influences the effectiveness of gene transfer by plasmid DNA. Multiple injections may improve the overall expression by a single target organ or tissue, but can also result in deleterious side effects such as induction of host-mediated immune response. Fewer injections are preferable and can range from 1-20, 1-15,1 -10 or 1-5.

The non-viral carriers for the gene are preferably those compounds that are known to have a long half-life in the blood. As a result of this relatively long half-life in the blood, the target cell is exposed for as long as possible to as high a concentration of the vector as possible in order thereby to achieve the maximum possible binding of vectors to the target cells by means of the target cell-specific ligands. In a particularly preferred embodiment, these non-viral carriers are cationized to enable them to complex with the negatively charged DNA.

The non-viral carriers for the gene, that can be employed in accordance with the invention, are known in the art (for reviews, see Cotten et al., Curr. Biol., 4: 705 (1993); Behr, Acc. Chem. Res., 26: 274 (1993); Felgner, Adv. Deliv. Rev., 5: 163 (1990); Behr, Bioconjugate Chem., 5: 382 (1994); Ledley, Hum. Gene Ther. 6: 1129 (1995) all of which are hereby incorporated by reference). Those that are preferably employed within the context of the present invention are liposomes, cationic liposomes that are prepared using cationic lipids such as stearyl amines in a mixture with neutral phospholipids, dioctadecyldimethylammonium bromide (DDA) in a mixture with neutral phospholipids, N-1(2,3-dioleyloxy)propyl-N,N,N-trimethylammonium bromide(DOTMA), 3. beta. -N-(N′,N′-dimethylaminoethane)carbamoyl cholesterol (DC-Chol), 1,2-dimyristyloxypropyl-3-dimethylhydroxyethylammonium bromide (DMRIE), dimethyldioctadecylammonium bromide (DDAB) and 1,2-dioleoyloxy-3-(trimethylammonio)propane (DOTAP).

Cationic polypeptides and proteins, such as polylysine, protamine sulfates, histones, polyornithine and polyarginine, are also suitable as non-viral carriers, as are cationic amphiphilic lipopolyamines such as dioctadecylamidoglycylspermine (DOGS), dipalmitoylphosphatidylethanolamidospermine (DPPES), N-t-butyl-N′-tetradecyl-3-tretradecylaminopropionamide (diC14-amidine), DOTB, ChoTB, DoSC, ChoSC, LPLL, DEBDA, DTAB, TTAB, CTAB or TMAG, or cationic polysaccharides such as diethylaminoethyldextran, and also cationic organic polymers such as Polybrene.

In a preferred embodiment, liposomes are prepared from phosphatidyl serine where stock lipids are dried onto the surface of a plastic dish and allowed to dry. Ten ml of 0.3 M sucrose is layered, and the liposomes allowed to bubble into the bulk phase over a 1 hour period. The sucrose solution is recovered and mixed 1:1 with 10 ml of PBS followed by centrifugation at 15,000 rpm for 20 min at 4° C. The liposome pellet is resuspended in a 1 ml volume of 150 mM NaCl and 25 mM Tris-HCl, pH 7.5. The concentration of the final liposome solution is determined by phosphate determination and adjusted accordingly.

There are a number of natural activators and inhibitors of Factor XIIa, which may influence the intrinsic activity of Factor XIIa when present in a subject for an extended period of time. The physiological activation of Factor XII occurs by exposure to anionic surfaces, such as found on cut vascular elements (Griffin and Cochrane, 1976), or by subendothelial collagen. However, collagens III, IV and V inhibit Factor XII activation (Koenig et al, 1991). The activation of Factor XII to XIIa is blocked by amyloid precursor protein (APP) isoforms, with and without the Kunitz protease inhibitor domain (Niwano et al, 1995). These forms of APP are secreted by activated platelets, T-lymphocytes and leukocytes. Antithombin III is a plasma inhibitor of Factor XIIa (Chan et al, 1977). However, Pixley et al (1985) report that antithrombin III only contributes 2-3% to the total inhibition of Factor XIIa by plasma, and that the predominant inhibitor is C1 inhibitor. Estrogens also enhance Factor XII levels in the plasma, and may be a reason for a hypercoagulable state induced in some patients on estrogen therapy (Citarella et al, 1996). Bismuth subgallate, a known activator of Factor XII to XIIa, can reduce the clotting time of plasma deficient in Factor VII (Thorisdottir et al, 1988).

A primary object is to provide a method of gene therapy where the expressed protein is secreted directly into the blood stream to obtain therapeutically effective amounts of the protein thereby treating the subject in need of the protein.

The term “effective amount” means a sufficient amount of compound, e.g. nucleic acid and its corresponding recombinant protein delivered to produce an adequate level of the FXII, i.e., levels capable of converting VII to VIIa. Thus, the important aspect is the level of FXII expressed. Accordingly, one can use multiple transcripts or one can have the gene under the control of a promoter that will result in high levels of expression. In an alternative embodiment, the gene would be under the control of a factor that results in extremely high levels of expression.

Coagulation activity in the blood plasma of a recipient is determined by bypass activity following administration of a recombinant FXIIa-coated bead, an implant containing a recombinant FXIIa-coated bead, a cell expressing recombinant FXII, an implant containing a cell expressing recombinant FXII, a naked DNA vector containing a gene encoding a FXII polypeptide or a truncated or mutated form thereof, or a liposome encapsulated DNA vector containing a gene encoding a FXII polypeptide or a truncated or mutated form thereof.

Bypass activity is measured by the activated partial thromboplastin time (aPPT) assay. An aPPT assay for the quantification of FVII bypass activity is performed by preincubation of a mixture containing 100 μl of Factor VIII-deficient plasma, 100 μl of aPPT reagent (Sigma), and 20 μl of test samples for 3 min at 37° C. in a Fibrometer fibro-cup (B&L Fibro systems, Cockeysville, Mo.). The reaction is initiated by the addition of 100 μl of 25 mN CaCl₂. The time taken for formation of the clot is determined, and a value in units s⁻¹ is calculated from the reciprocal of the clotting time. Control measurements are made from pooled normal plasma.

The present invention is described in further detail in the following experimental details section, which sets forth specific examples to aid in understanding the invention and should not be construed to limit in any way the invention as defined in the claims which follow thereafter.

### EXAMPLE 1

Ex-vivo and in vivo Gene Therapy Systems for Delivery of Recombinant, Full Length or modified Factor XII.

As an alternative to introducing natural abundance or recombinant Factor XIIa within a porous chamber implant, it is also possible to introduce full length or modified Factor XII directly into the recipient using all the same gene therapy systems traditionally employed for gene therapy of individual Factor VIII or IX genes. Factor XII gene therapy is a universal therapeutic for both Hemophilia A and B.

Immobilized, recombinant Factor XIIa was introduced directly into a recipient, e.g., a guinea pig, and the bypass activity generated over time was measured by the aPPT assay. Guinea pigs were obtained, housed and sampled as described (Nature Biotechnology 18:289-295, March 2000) except that a 0.5 ml sample of Factor XIIa beads (50% slurry in PB) was injected, intraperitoneally, into guinea pigs. Glycine beads were used a negative control. Samples of blood were obtained by cardiac puncture one day prior to the injections in the guinea pigs. Thereafter, blood samples were obtained by the same route at 24, 48, and 72 hours post injection. A peak of bypass activity was noted at 24 hours post injection in guinea pigs injected with Factor XIIa beads, but not with the glycine bead control. Bypass activity was back to baseline by 48 hours (data not shown). No changes in activity were noted at other times under either condition.

Introduction of Factor XIIa into the peritoneal cavity, unprotected by the membrane-enclosed porous chamber, was transiently therapeutic. It follows that a method of sustainedly expressing Factor XIIa activity might also prove therapeutic over a sustained period of time. The advantages of such a method would be (i) that therapy would be effective for both Hemophilia A and B; (ii) that antibodies against Factor XII would be unlikely since the factor is present at some level in most hemophiliacs; (iii) that antibodies against Factor VIII would not be generated; and (iv) that biological strategies to decrease immune responses would not be needed.

### EXAMPLE 2

### Treatment of Congenital Factor XII Deficiency

It has been reported that some patients with either Hemophilia A or B also have a congenital partial lack of Factor XII (Barthels et al, 1982). Indeed, there is a significant prevalence of moderate to severe Factor XII deficiency among the general European population, amounting to 2.3% (Halbmayer et al, 1994). Fortunately, the consequence of Factor XII deficiency alone is not profound. A case of a Japanese girl has also been reported with both hemophilia A and heterozygous Factor XII deficiency (Matsushita et al, 1992). The incidence of heterozygous Factor XII deficiency is not known in any population, nor was the genotype reported by Halbmayer et al (1994). Accordingly, any one or a combination of the aforementioned embodiments for presenting a recombinant FXII polypeptide can be applied in gene therapy related methods for treating patients with congenital deficiencies for FXII.

### REFERENCES

Andrews, J. L., Weaver, L., Kaleko, M., and Connelly, S. (1999) Efficient adenoviral vector transduction and expression of functional human factor VGIII in cultured primary human hepatocytes. Hemophilia 5:160-168.

Balague, C., Zhou, J., Dai, Y., Alemany, R., Josephs, S. F., Andreason, G., Hariharan, M., Sethi, E., Prokopenko, E., Jan, H. Y., Lou, Y. C., Hubert-Leslie, D., Ruiz, L., Zhang, W. W. (2000) Sustained high level expression of full length factor VIII and restoration of clotting activity in hemophiliac mice using a minimal adenovirus vector. Blood 95:820-828.

Bandyopadhyay, P., Ma, X., Linehan-Stierrs, C., Kren, B. T., and Steer, C. J.(1999) Nucleoitideexchange in genomic DNA or rat hepatocytes using RNA/DNA oligonucleotides. Targeted delivery of liposomes and polyethyleneimine to the asialoglycoprotein receptor. J.Biol.Chem. 274:10163-10172.

Barthels, M., Edel, J., Liese, B., and Karges, H. E. (1982) Additional factor XII (Hageman factor) deficiency in hemophilia A and in von Willebrand syndrome.Klin.Wochenschr 60:303-309.

Burton, M., Nakai, H., Colosi, P., Cunningham, J., Mitchett, R., and Couto, L. (1999) Coexpression of factor VIII heavy and light chain adeno-associated viral vectors produces biologically active protein Proc.Nat.Acad.Sci.(USA) 96:12725-12730.

Chan, J. Y., Burrowes, C. E., Habal, F. M., and Movat, H. Z. (1977) The inhibition of activated factor XII (Hageman factor) by antithrombin III: the effect of other plasma proteinase inhibitors. Biochem. Biophys. Res. Commun. 74:150-158.

Chiang, G. G., Rubin, H. L., Cherington, V., Wang, T., Soboewski, J., McGrath, C. A., Gaffney, A., Emami, S., Sarver, N., Levine, P. H., Greensberger, J. S., and Hurwitz, D. R. (1999) Bone marrow stromal cell-mediated gene therapy for hemophilia A: in vitro expression of human factor VIII with high biological activity requires the inclusion of the proteolytic site at amino acid 1648. Hum.Gene Therap. 10:61-76.

Citarella, F., Misiti, S., Felici, A., Farsetti, A., Pontecorvi, A., and Fantoni, A. (1996) Estrogen induction and contact phase activation of human factor XII. Steroids 61:270-276.

Connelly, S., Andrews, J. L., Gallo, A. M., Kayda, D. B., Qian, J., Hoyer, L., Kadan, M. J., Gorziglia, M. I., Trapnell, B. C., McClelland, A., and Kaleko, M. (1998) Sustained phenotype correction of murine hemophilia A by in vivo gene therapy. Blood 91:3273-3281.

Evans, G. L., and Morgan, R. A. (1998) Genetic induction of immune tolerance to human clotting factor VIII in a mouse model for hemophilia A. Proc.Nat.Acad.Sci.(USA) 95:5734-5739

Gnatenko, D. V., Saenko, E. L., Jesty, J., Cao, L. X., Hearoing, P., and Bahou, W. F. (1999) Human factor VIII can be packaged and functionally expressed in an adeno-associated virus background: applicability nto haemophilia A gene therapy. Br.J.Haematol. 104:27-36.

Griffin, J. H. and Cochrane, C. G. (1976) Human Factor XII (Hageman Factor) Methods in Enzymol. 45:56-65.

Herzog, R. W., Yang, E. Y., Couto, L. B., Hagstrom, J. N., Elwell, D., Fields, P. A., Burton, M., Bellinger, D. A., Read, M. S., Brinkhous, K. M., Podsakoff, G. M., Nichol, T. C., Kurtzman, G. J., and High, K. A. (1999) Long-term correction of canine hemophilia B by gene transfer of blood coagulation factor IX mediated by adeno-associated viral vector. Nature Medicine 5:21-22.

Kaufman, R. J. (1999) Advances toward gene therapy for hemophilia at the millennium. Hum.Gene Ther. 10:2091-2107.

Koenig, J. M., Chahine, A., and Ratnoff, O. D. (1991) Inhibition of the activation of Hageman factor (factor XII) by soluble human placental collagens types III, IV and V. J.Lab C1in.Med. 117:523-527.

Lacroix-Desmazes, S., Moreau, A., Sooryannarayana, ., Bonnemain, C., Stieltjes, N., Pashov, A., Sultan, Y., Hoebeke, J., Kazatchkine, M. D., and Kaveri, S. V. (1999) Catalytic activity of antibodies against factor VIII in patients with hemophilia A. Nature Medicine 5:1044-1047.

Matsushita, T., Takamatsu, J., Kagami, K., Takahashi, I., Sugiura, I., Hamaguchi, M., Kamiya, T. and Saito, H. (1992) A female hemophilia A combined with hereditary coagulation factor XII deficiency: a case report. Am.J.Hemat. 39:137-141.

Monahan, P. E., Samulski, R. J., Tazelaar, J., Xiao, X., Nichols, T. C., Bellinger, D. A., Read, M. S., and Walsh, C. E. (1998) Direct intramuscular injection of recombinant AAV vectors results in sustained expression in a dog model of hemophilia. Gene Therap. 5:40-49.

Niwano, H., Embury, P. B., Greenberg, B. D., and Ratnoff, O. D. (1995) Inhibitory action of amyloid precursor protein against human Hageman factor (factor XII). J.Lab.Clin.Med. 125:251-256.

Oh, S. H., Kim, S. H., Kim, H. W., and Kim, Y. J. (1999) An efficient retrovirus-mediated transduction of human blood coagulation factor VIII cDNA in regeneration rat liver. Ann.Hematol. 78:213-218.

Pipe, S. W. and Kaufman, R. J. (2000) A chamber of hope for hemophilia. Nature Biotechnology, 18: 264-265.

Pixley, R. A., Schapira, M., and Colman, R. W. (1985) Effect of heparin on the inactivation of human activated factor XII by antithrombin III. Blood 66:198-203.

Prescott, R., Nakai, H., Saenko, E. L., Scharrer, I., Nilsson, I. M., Humphries, J. E., Hurst, D., Bray, G., and Scandella, D. (1997) The inhibitor antibody response is more complex in hemophilia A patients than in most nonhemophiliacs wit factor VIII autoantibodies. Recombinate and Kogenate Study Groups. Blood 89:3663-3671.

Ton-That, T. T., Doron, D., Pollard, B. S., Bacher, J., and Pollard, H. B. (2000) In vivo bypass of hemophilia A coagulation defect by Factor XIIa implant. Nature Biotechnology, 18:289-295.

VandenDriessche, T., Vanslembrouck, V., Goovaerts, I., Zwinnen, H., Vanderhaegen, M. L., Collen, D., and Chuah, M. K. (1999) Long term expression of human coagulation factor VIII and correction of hemophilia A after in vivo retroviral gene transfer in factor VIII-deficient mice. Proc. Nat.Acad. Sci.(USA) 96:10379-10384.

Zatloukal, K., Cotton, M., Berger, M., Schmidt, W., Wagner, E., and Birnstiel, M. L. (1994) In vivo production of human factor VIII in mice after intrasplenic implantation of primary fibroblasts transfected by receptor-mediated adenovirus-augmented gene delivery. Proc.Nat.Acad.Sci.(USA) 91:5148-5152.

Zhang, W. W., Josephs, S. F., Zhou, J., Fang, X., Alemany, R., Belague, C., Dai, Y., Ayares, D., Prokopenko, E., Lou, Y. C., Sethi, E., Hubert-Leslie, D., Kennedy, M., Ruiz, L., and Rockow-Magnone, S. (1999) Development and application of a minimal-adenoviral vector system for gene therapy of hemophilia A. Thrombos.Haemost. 82:562-571.

What is claimed:

1. A factor XIIa-coated solid support comprising a recombinant factor XII polypeptide:

2. The Factor XIIa-coated solid support of claim 1, wherein the polypeptide is a truncated polypeptide.

3. The Factor XIIa-coated solid support of claim 2, wherein the truncated polypeptide comprises a sequence of amino acid residues 1-215 of SEQ ID No. 2.

4. The Factor XIIa-coated solid support of claim 2, wherein the truncated polypeptide comprises a sequence of amino acid residues of about 1-196 of SEQ ID No. 2.

5. The Factor XIIa-coated solid support of claim 1, wherein the solid support is a bead.

6. A Factor XII-expressing cell comprising an expression vector construct containing a gene encoding a Factor XII polypeptide.

7. The Factor XII-expressing cell of claim 6, wherein the polypeptide is a truncated polypeptide.

8. The Factor XII-expressing cell of claim 7, wherein the truncated polypeptide comprises a sequence of amino acid residues 1-215 of SEQ ID No. 2.

9. The Factor XII-expressing cell of claim 7, wherein the truncated polypeptide comprises a sequence of amino acid residues of about 1-196 of SEQ ID No. 2.

10. The Factor XII-expressing cell of claim 6, wherein the cell comprises a blood cell, a hepatocyte, a fibroblast, and an endothelial cell.

11. A method of treating a subject with a coagulation protein comprising providing a gene prepared as a naked DNA vector and administering the vector to the subject in a therapeutically effective amount, wherein the gene encodes a Factor XII polypeptide.

12. A method of treating a subject with a coagulation protein comprising the steps of
a) providing a gene for a coagulation protein prepared as an expression vector construct;
b) transfecting host cells with the construct,
c) adding cells of step b) expressing the coagulation protein to a porous chamber; and
d) implanting the chamber into the subject, wherein the chamber is implanted in fluid communication with the bloodstream and inactive Factor VII in the bloodstream passing through the chamber becomes activated upon contact with the coagulation protein.

13. The method of claim 12, wherein the coagulation protein is a Factor XII polypeptide.

14. The method of claim 13, wherein the polypeptide is a truncated polypeptide.

15. The method of claim 14, wherein the truncated polypeptide comprises a sequence of amino acid residues of about 1-196 of SEQ ID No. 2.

16. The method of claim 12, wherein the chamber comprises a permeable membrane having one or more walls, defining a hollow chamber therewithin, a plurality of holes extending through the membrane and permitting fluid to enter and exit the membrane, each of the holes being sized so that it is just large enough to permit Factor VII to enter the chamber of the membrane, and activated Factor VIIa to exit the chamber, and an effective amount of the coagulation protein or a source of the coagulation protein being expressed by the cells.

17. The method of claim 16, wherein the permeable membrane is a dialysis membrane formatted as a geometric filter comprising polymethacrylate or polycarbonate, or a statistical hole filter comprising cellulose acetate.

18. The method of claim 12, wherein the chamber comprises a region for affixing the chamber to an abdominal wall and for removing or adding materials such as a diaphragm for penetration of a syringe.

19. The method of claim 12, wherein the expression vector construct of a) is a naked DNA.

20. The method of claim 12, wherein the expression vector construct of a) is encapsulated by a liposome.

21. The method of claim 12, wherein the expression vector construct of a) is a viral vector.

22. A method of treatment with a coagulation protein comprising administering to a subject, a therapeutically effective amount of a recombinant Factor XII polypeptide.
